(19) United States
(12) Patent Application Publication
(10) Pub. No.: US 2008/0195223 A1
(43) Pub. Date: Aug. 14, 2008

(54) Materials and Methods and Systems for Delivering Localized Medical Treatments

(76) Inventors: Avram Allan Eddin, Portola Valley, CA (US); Robert Wenz, Wollstadt (DE); Jorg Meyer, Heusensstamm (DE)

Correspondence Address: KILPATRICK STOCKTON LLP - 55461, 1001 WEST FOURTH STREET, WINSTON-SALEM, NC, 27101, US

(21) Appl. No.: 11/933,618

(22) Filed: Nov. 1, 2007

Related U.S. Application Data
(60) Provisional application No. 60/856,437, filed on Nov. 3, 2006.

(51) Int. Cl. A61F 2/28; A61B 17/58

(52) U.S. Cl. 623/23.62; 606/92

(57) ABSTRACT

A filler material comprising an energy intercepting element. In an embodiment, an energy intercepting element may attenuate, redirect, concentrate, or otherwise modify the properties of intercepted energy, e.g. the incoming energy flux. In an embodiment an energy intercepting element may be changed, for example generating secondary electrons, and/or secondary radiation, by intercepted energy. Also disclosed is a method comprising introducing a filler material composition into a cavity within a tissue in a patient's body, wherein the filler material includes an energy intercepting element(s) for intercepting energy and directing an energy source at the filler material.

### STATEMENT OF RELATED APPLICATIONS

The present application claims priority under 35 USC 119 from U.S. Provisional Application Ser. No. 60/856,437, filed Nov. 3, 2006, entitled “Materials and Methods and Systems for Delivering Localized Medical Treatments” the disclosure of which is hereby incorporated herein by reference.

### FIELD OF THE INVENTION

The present invention relates to compositions useful in the medical, veterinary and/or dental arts for filling voids. The present invention also relates to methods and systems for delivering localized medical treatments.

### BACKGROUND OF THE INVENTION

Fractured vertebral bodies can be successfully treated by balloon-kyphoplasty. In a balloon-kyphoplasty treatment a balloon is placed within a fractured vertebral body and inflated to create a cavity that may be filled with a cement composition. The cement composition augments the bone and provides structural support. Similar balloon techniques, or other techniques such as drilling, may be utilized to create cavities in other bone structures to fill with a cement composition.

Tumors, such as metastatic tumors found in cancer, may weaken bone structures. For example, tumor located near, and especially in, bone structures may cause the bone structures to weaken and/or fracture. Although such fractures may be treated and/or stabilized with techniques such as balloon-kyphoplasty, in many cases after the balloon-kyphoplasty treatment there is still a need for subsequent repeated energy based (e.g. radiation) and/or pharmacological (e.g. chemotherapy) treatment.

It would be desirable to have a bone filler (e.g., cement composition, etc.) that may be used in existing and/or new medical, veterinary and/or dental procedures to provide structural support to the weakened bone, and that could also be used in a therapeutic treatment for tumors and other medical conditions. Ideally, the same novel bone filler material could also be used ab initio to treat tumors and other medical conditions, independent of and/or prior to structural deficiency of the bone itself

### SUMMARY OF THE INVENTION

In one variation, the present invention provides filler material compositions comprising an energy intercepting element. In an embodiment, an energy intercepting element may attenuate, redirect, concentrate, or otherwise modify the properties of intercepted energy, e.g. the incoming energy flux. In an embodiment an energy intercepting element may be changed, for example generating secondary electrons, and/or secondary radiation, by intercepted energy.

In an embodiment, a filler material composition, suitable for use in medical, veterinary and/or dental applications to provide structural support, comprises an energy intercepting element.

In an embodiment, a filler material composition comprises a carrier material for an energy intercepting element. In an embodiment, a filler material comprises a cement composition.

In an embodiment, an energy intercepting element generates heat upon intercepting energy.

In an embodiment, an energy intercepting element generates electrons intercepting energy.

The present invention also provides methods for providing hyperthermic treatment comprising introducing a filler material composition comprising an energy intercepting element to a location near a location where hyperthermic treatment is desired, and exposing the energy intercepting element to energy thereby raising the temperature of the energy intercepting element.

In another variation, in an embodiment a method comprises introducing a filler material composition into a cavity within a tissue in a patient's body, wherein the filler material includes an energy intercepting element(s) for intercepting energy. An energy source (e.g., electromagnetic wave, electric field, magnetic field, high energy particle field, microwave, high-frequency light ray, X-ray, electron-beam, etc.) is then directed at the filler material. In an embodiment, at least a portion of the energy projected by the energy source is intercepted by an energy intercepting element in the filler material. The energy may be redistributed around and/or throughout the filler material. In an embodiment, the redistribution may be substantially uniform. In an embodiment, the redistribution may comprise localized portions reacting to a greater or lesser extent to the energy. The redistributed energy may induce a therapeutic effect on the tissue surrounding the filler material. For example, the redistributed energy may interferer with cancerous cells residing in tissue surrounding the filler material (e.g., destroys cancerous cell and/or inhibits cancerous cell growth).

### DETAILED DESCRIPTION OF THE INVENTION

For the purposes of this specification, unless otherwise indicated, all numbers expressing quantities of ingredients, reaction conditions, and so forth used in the specification are to be understood as being modified in all instances by the term “about.”Accordingly, unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

In one variation the present invention provides bone filler material compositions, such as cement compositions, and methods for providing localized treatment (e.g. hyperthermic treatment). In an embodiment, a method of the present invention includes the use of a composition of the present invention. Methods of the present invention, however, are not limited to use of compositions of the present invention, but may also use other compositions. Similarly, an embodiment of a filler material composition of the present invention may be useful and advantageous for performing a method of the present invention. Filler material compositions of the present invention, however, are advantageous for many other uses known in the art for filler materials and/or cement compositions.

In an embodiment, a filler material composition of the present invention comprises an energy intercepting element element. The energy intercepting element may comprise a percentage, by weight, of the filler material composition sufficient to intercept energy when the filler material composition is exposed to an energy source. In an embodiment, the energy intercepting element comprises a percentage, by weight, of the filler material composition sufficient to intercept sufficient energy to generate an amount of heat sufficient for a hyperthermic treatment of a location. In an embodiment the energy intercepting element is capable of generating sufficient heat to kill cancerous cells.

In an embodiment, the energy intercepting element comprises a metal, ceramic, polymer or other element capable being excited upon intercepting energy to produce heat. Suitable metals include periodic table group III-XII elements including, but not limited to, titanium, tungsten, iron, palladium, cobalt, nickel, copper, zinc, gold, silver, platinum, and/or oxides and/or alloys thereof Suitable ceramics and polymers include ceramic and/or polymers that are capable of being excited by a source. In an embodiment, a cement composition, upon hardening, is capable of receiving multiple exposures to a source with minimal loss of strength and other physical properties.

In an embodiment, an energy intercepting element comprises iron (Fe) and/or iron oxide (Fe(II,III)₃O₄). In another embodiment, an energy intercepting element comprises a palladium (Pd)/cobalt (Co); Co/Platinum(Pt) or Fe/Pd alloy. In another embodiment, an energy intercepting element comprises tungsten (W).

In an embodiment an energy intercepting element may be selected in view of the desired use the filler material composition. As discussed above, an energy intercepting element may attenuate, redirect, concentrate, or otherwise modify the properties of intercepted energy. An energy intercepting element may also be modified by intercepted energy. In an embodiment the choice of energy intercepting element may be made based on the desired affect the energy intercepting element will have on the energy intercepted.

In an embodiment, a filler material further comprises a carrier material for the energy intercepting element. In an embodiment, a carrier material comprises a structural element.

In an embodiment, a filler material comprises ionic bonds between at least a portion of a carrier material and at least a portion of an energy intercepting element. In an embodiment, a fiber material comprises covalent bonds between at least a portion of a carrier material and at least a portion of an energy intercepting element. In an embodiment, a filler material comprises an energy intercepting element in suspension in a carrier material. In an embodiment, a filler material comprises an energy intercepting element aggregate. In an embodiment, a filler material comprises ionic bonds between at least a portion of a carrier material and at least a portion of an energy intercepting element; covalent bonds between at least a portion of a carrier material and at least a portion of a energy intercepting element; an energy intercepting element in suspension in a carrier material and/or an energy intercepting element aggregate.

In an embodiment a carrier material comprises a biocompatible polymer. In an embodiment a carrier material is initially flowable and capable of setting to a sufficiently hardened state to maintain an energy intercepting element in a desired position.

In an embodiment, a filler material composition comprises an acrylic cement. In another embodiment, a filler material composition comprises an inorganic cement.

In an embodiment, a filler material composition is injectable. In an embodiment a filler material composition is configured to be delivered through a cannula into a void in a fluid or semi-fluid state, and thereafter solidifies into a solid.

The additional components of a filler material composition of the present invention may include the components typically found in acrylic and/or inorganic bone cements. Examples of cement composition formulations, suitable for use in an embodiment of the present invention, are described in U.S. Pat. Nos. 7,115,163; 4,612,053; 5,149,368; 4,518,430; 6,641,587; 6,599,520; 6,075,067; 6,375,659; PCT Publications WO 96/14265; EP Publications EP 1 296 909 A1 and EP 0 835 668 A1, and U.S. Patent Publication Nos 20050287071; 20050142211. The full disclosures of each of the patents and published applications are incorporated herein by reference in their entirety for all purposes.

The relative amounts of an energy intercepting element, or elements, and filler material may be varied to produce a composition with desired properties for a particular end use. The amount, by weight, of energy intercepting element(s) and filler material, and the compositions of each, will affect the amount of energy intercepted and other properties the composition. The energy intercepting element may comprise a percentage by weight, of the filler material composition sufficient to intercept energy when the filler material composition is exposed to an energy source. In an embodiment, the energy intercepting element comprises a percentage, by weight, of the filler material composition sufficient to intercept sufficient energy to generate an amount of heat sufficient for a hyperthermic treatment of a location.

In an embodiment, a composition comprises 10 to 90%, by weight, an energy intercepting element, or combinations of energy intercepting elements and 90 to 10% a cement. In an embodiment, a composition comprises 20 to 80%, by weight, an energy intercepting element, or combinations of energy intercepting elements and 80 to 20% a cement. In an embodiment, a composition comprises 30 to 70%, by weight, an energy intercepting element, or combinations of energy intercepting elements and 70 to 30% a cement. In an embodiment, a composition comprises 40 to 60%, by weight, an energy intercepting element, or combinations of energy intercepting elements and 60 to 40% a cement.

In an embodiment, the strength of a filler material composition should be sufficient for the intended use. The strengths of a filler material composition can be increased by adding reinforcing materials. The amount that is added is dependent on where the filler material composition is to be used. If used within bones, the amount of reinforcing material may depend on whether or not those bones are load bearing bones. In an embodiment wherein a filler material is used as part of a supporting structure, for example as a void filler in a vertebral body, the filler material should have sufficient strength to provide support to the vertebral body. In an embodiment a filler material for use in vertebral bones has a strength of 2-12 MPa. In another embodiment, a filler material for use in a long bone may have a strength of 25 to 90 MPa. In an embodiment, a filler material has a strength of 0.5 to 100 MPa. In an embodiment, a filler material has a strength of 0.5 to 12.0 MPa. In an embodiment, a filler material has a strength of 0.5 to 5.0 MPa. In an embodiment, a filler material has a strength of 0.5 to 2.5 MPa.

In an embodiment a filler material further comprises reinforcing materials. In an embodiment a reinforcing material forms part of a matrix, for example, an organic compound such as PMMA over an inorganic calcium salt. In an embodiment a reinforcing material comprises particulate matter. In an embodiment, particulate matter comprises metallic particulate matter, ceramic particulate matter and/or polymeric particulate matter.

In an embodiment, a cement comprises an energy intercepting element. In effect, in an embodiment, a cement is capable of intercepting energy as part of its matrix.

In an embodiment, a filler material composition is injectable. In an embodiment, a filler material composition may be used in a balloon kyphoplasty procedure, or a bone augmentation procedure, such as a procedure described in U.S. Pat. Nos. 7,044,954; 6,899,719; 6,814,736; 6,726,691; 6,719,773; 6,719,761; 6,716,216 and/or U.S. Patent Publication Nos. 20060149281; 20050119662; 20040210231; 20040167562; 20040049203; 20040024409;20040010263; 20040010260; 20020161373; 20020156482; 20020099385; 20020099384; 20020082608; 20020082605; 20020049448. The full disclosures of each of the patents and published applications are incorporated herein by reference in their entirety for all purposes

Suitable sources for exciting a energy intercepting element include, but are not limited to: a magnetic field, microwaves, radiation, light, mechanical agitation or other means. In an embodiment, a source comprises a magnetic field. In another embodiment, a source comprises microwaves. In another embodiment, a source comprises an electron beam.

In an embodiment, a filler material composition is prepared by mixing a energy intercepting element, for example in particulate form, into a resin, for example an acrylic resin. The particle size of the energy intercepting element may be modified and selected such that the method and/or amount of energy intercepted is controlled. For example, in an embodiment, for a ferromagnetic energy intercepting element, particle size may be selected such that heat is produced by either a faraday current (eddy current) or due to hysteresis loss of the magnetic orientation in individual particles as the field is cycled. In an embodiment, a energy intercepting element may be selected such that a specific Curie temperature, defined by the energy intercepting element, is reached upon excitation thereby allowing control over heat production not only by the application of the external energy that excites the energy intercepting element, but the energy intercepting element itself.

The present invention also provides methods for treating medical conditions, including tumors. In an embodiment, a method of the present invention comprises placing a filler material composition comprising a energy intercepting element near a site to be treated and exciting the energy intercepting element to generate heat. Exciting the energy intercepting element may comprise subjecting the energy intercepting element to a magnetic force microwaves, radiation, light, mechanical agitation, thermal heating or other means. In an embodiment, heat generated by exciting the energy intercepting element is received by the tumor providing a hyperthenmic treatment to the tumor.

Hyperthennic treatment of musculoskeletal tumors are described in Onkology 44:1-5, 1987. Tumor cells Generally have a higher susceptibility to temperature than normal tissue and have shown to be treatable by hyperthermic treatment.

In an embodiment of the present invention, a filler material composition is located sufficiently near a site to receive hyperthernic treatment, such as, for example, a tumor, such that exciting a energy intercepting element in the cement will generate sufficient heat to enable the hyperthermic treatment at the desired site, via conduction, convection or radiation.

The step of locating a filler material composition near a site to be treated may be accomplished by injecting the filler material composition and/or by other techniques known in the art for delivering cement compositions to a site. In an embodiment, a filler material composition is located near a site to be treated utilizing one or more of the techniques described in the afore-mentioned patents and/or patent publications.

In an embodiment, after locating the filler material composition, the filler material composition is subjected to an external force/energy sufficient to excite the energy intercepting element component(s) of the composition. The external force/energy may be generated and/or delivered by means known in the art, for example using techniques similar to those used in magnetic resonance imaging to excite the energy intercepting element through the application of a magnetic field. In an alternative embodiment a energy intercepting element may be excited through the application of radiation, such as X-ray or microwave radiation.

In an embodiment, the excitation may occur through application of an energy external to the site of the filler material composition. For example, a magnetic field may be generated external to a patient and pass through surrounding tissue to excite a energy intercepting element at a desired treatment site.

In an embodiment, a focusing device is utilized to focus energy emitted by an external energy source onto the filler material composition embedded in a tissue inside the patient's body. Radiographic techniques or other imaging modalities may also be implemented to located the position of the filler material composition and direct the focused energy on to the filler material composition. In one variation, an x-ray radiographic device is utilized to locate the position of the filler material composition in a vertebral body. An electromagnetic wave source and a corresponding focusing device is then aligned and focused onto the filler material composition. A focused energy is directed onto the filler material composition. Particles and/or elements in the filler material composition intercept (e.g., absorbs, reflects, refracts deflects, etc.) the focused energy, and redirect and/or release the energy onto tissue surrounding the filler material composition which may result in therapeutic effect on the tissue.

In an embodiment of the present invention, the step of exciting the energy intercepting element may be repeated multiple times during a single treatment session, or over multiple treatment sessions.

In an embodiment, the step of exciting the energy intercepting element may be continuous over a period of time, with an energy source affixed to the patient so as to provide mobility during a treatment session.

In an embodiment, a method of the present invention comprises: creating a void in a bone structure; injecting a filler material of the present invention, in a fluid or semi-fluid state, into the void through a cannula; allowing the filler material to solidify; and exciting an energy intercepting element in the filler material.

In an embodiment, a void may be created by compacting cancellous bone in a bone structure.

In an embodiment the bone structure comprises a vertebral body.

In an embodiment the void is located within a bone structure, the bone structure comprises a cortical bone and a cancellous bone and the filler material when solidified is capable of bearing loads.

In an embodiment, the void comprises at least a portion of a vertebral body in a patient's spine, the void is created by compacting at least portion of a cancellous bone in the vertebral body, the filler material when solidified inside the void is capable of bearing loads when the patient stands upright.

The present invention also provides systems. In an embodiment, a system of the present invention comprises a filler material composition of the present invention. A system may further comprise surgical tools, for example cannula, tamping instruments and the like described in the afore-referenced patents and patent publications for delivering a filler material composition to a site in a patient. In addition, or in the alternative, a system may further comprise surgical tools for creating a void space within a patient's body, such as the surgical tools that are also described in the afore-referenced patents and patent publications. In an embodiment, a system may comprise an apparatus for exciting a energy intercepting element in a filler material composition.

### EXAMPLE

A filler material composition of the present invention comprising a powder/liquid system was prepared by admixing 20.0 grams of a mixture of 50%, by weight, size 150 mm iron beads and 50%, by weight, MW 422 acrylic resin beads containing 2%, by weight benzoyl peroxide (BPO); 3.5 grams methyl methacrylate copolymer (MMA) containing 5%, by weight butyldimetharylate (BDMA) and 1%, by weight, N,N di-methyl-para-toluidine (DMpT).

The resulting composition, when hardened, is subjected to a high frequency alternating magnetic field of 5-10 kV and a frequency of 100 kHz generated by a device produced by Huettinger Elektronik GmbH and Co., Freiburg, Germany. The magnetic field results in the composition attaining a temperature suitable for hyperthermic treatment.

1. A filler material comprising an energy intercepting element.

2. The filler material of claim 1, further comprising a carrier material.

3. The filler material of claim 1 further comprising a cement.

4. The filler material of claim 2, wherein the filler material is configured to be delivered through a cannula into a bone in a fluid or semi-fluid state, and thereafter solidifies into a solid.

5. The filler material of claim of claim 1 wherein the energy intercepting element comprises: a metal, ceramic, or polymer.

6. The filler material of claim 5 wherein the metal comprises: a group III-XII element; an oxide of a group III-XII element; and/or an alloy comprising a group III-XII element.

7. The filler material of claim 6 wherein the metal comprises: tungsten, titanium, iron, palladium, cobalt, nickel, copper, zinc, gold, silver, platinum, and/or oxides and/or alloys thereof.

8. The filler material of claim 6 wherein the metal comprises: iron (Fe) or iron oxide (Fe(II,III)₃O₄).

9. The filler material of claim 6 wherein the metal comprises a palladium (Pd)/cobalt (Co); Co/Platinum(Pt) or iron(Fe)/palladium(Pd) alloy.

10. The filler material of claim 1 further comprising an acrylic resin component.

11. The filler material of claim 10 wherein the filler material comprises 10 to 90%, by weight, the energy intercepting element.

12. The filler material of claim 11 wherein the filler material further comprises 90 to 10%, by weight the acrylic resin component.

13. A bone cement comprising the filler material of claim 1.

14. A system comprising a surgical tool and a filler material of claim 1.

15. The system of claim 14 further comprising a plurality of surgical tools.

16. The system of claim 15 wherein the plurality of surgical tools comprise a cannula and a tamping instrument.

17. A method comprising:
injecting a filler material comprising an energy intercepting element near a treatment site; and
exposing the filler material to energy.

18. The method of claim 17 wherein the energy comprises: a magnetic force, microwaves, electron beam, radiation, light, mechanical agitation or other means.

19. The method of claim 17 wherein the treatment site comprises a tumor.

20. The method of claim 19 wherein the energy intercepting element heats upon the applying of the energy.

21. The method of claim 19 wherein the energy intercepting element generates secondary radiation upon intercepting energy.

22. A method comprising:
creating a void near a treatment site;
injecting a filler material comprising an energy intercepting element into the void; and
applying an energy field to at least a portion of the filler material.

23. The method of claim 22 wherein the energy field comprises a high frequency alternating magnetic field.

24. The method of claim 23 wherein the magnetic field has a power of 0.5 to 15 kV and a frequency of 50 to 200 kHz.

25. The method of claim 24, wherein the treatment site comprises at least a portion of a vertebral body in a patient's spine, the void is created by compacting at least portion of a cancellous bone in the vertebral body, the filler material when solidified inside the void is capable of bearing loads when the patient stands upright.
